(19) United States
(12) Patent Application Publication
(10) Pub. No.: US 2026/0331289 A1
(43) Pub. Date: Oct. 22, 2026

(54) ARTIFICIALLY INTELLIGENT SYSTEM AND METHOD FOR PROGNOSTIC EVALUATION OF AUTOIMMUNE DISORDERS

(71) Applicant: Progentec Diagnostics, Inc., Oklahoma City, OK (US)

(72) Inventors: Eldon Jupe, Oklahoma City, OK (US); Gerald H. Lushington, Lawrence, KS (US); Melissa E. Munroe, Oklahoma City, OK (US); Mohan Purushothaman, Oklahoma City, OK (US)

(21) Appl. No.: 19/112,238

(22) PCT Filed: Sep. 18, 2023
(86) PCT No.: PCT/US2023/033066

§ 371 Date: Mar. 14, 2025

Related U.S. Application Data
(60) Provisional application No. 63/407,584, filed on Sep. 16, 2022.

(51) Int. Cl. G16H 50/30; G06F 40/20; G16H 10/40; G16H 10/60; G16H 20/10; G16H 40/67

(52) U.S. Cl. CPC G16H 50/30; G06F 40/20; G16H 10/40; G16H 10/60; G16H 20/10; G16H 40/67

(57) ABSTRACT

An artificially intelligent system and method for prognostic evaluation of autoimmune disorders. Certain objects and advantages of the present disclosure include methods and systems configured to aggregate and analyze clinical conversational data, patient biometric data, medical records data, blood biomarker test data, and patient biometric data to tune metrics that are increasingly specific to a patient in the creation of a dynamic patient phenotype to enhance clinical understanding of one or more factors that are specific to the patient's health status. One or more Al framework and engine may facilitate effective integration of communication-related insights into diagnostic and prognostic digital health resources. Exemplary systems, methods, and apparatuses according to the principles herein may comprise machine learning and deep learning techniques for the development of one or more quantitative metrics derived from clinical conversational data.

### CROSS-REFERENCE TO RELATED APPLICATIONS

This application claims priority benefit of U.S. Provisional Application Ser. No. 63/407,584 filed Sep. 16, 2022, and entitled “Novel Methods and Systems for Classification of Undiagnosed Persons at Risk of Autoimmune Conditions Using Innovative Virtual and Digital Models”; the entirety of which is hereby incorporated herein at least by virtue of this reference.

### FIELD

The present disclosure relates to the field of digital health systems and software as a medical device; in particular, an artificially intelligent system and method for prognostic evaluation of autoimmune disorders.

### BACKGROUND

According to current estimates by the National Institutes of Health, as many as 23.5 million persons in the United States may be afflicted with at least one autoimmune condition. But the actual size of the affected population in the United States is now estimated to be as high as 50 million people, according to the American Autoimmune Related Diseases Association (AARDA). The annual economic burden from autoimmune conditions on the United States healthcare system is estimated to be as high as $100 billion.

Diagnosis has remained a challenge for many of these patients. In the case of Systemic Lupus Erythematosus (SLE), the average time to diagnose is well over 7 years. Getting diagnosed can be a long and challenging process. While autoimmune diseases have unique characteristics, many of the observable symptoms, like fatigue and pain, overlap with more common conditions. As a result, clinicians work to rule out other health conditions before considering an autoimmune disease diagnosis. Additionally, laboratory tests are still being developed to test for specific autoimmune diseases. Current best practices, including antinuclear antibodies (ANA) tests, measure for the presence of a generic type of antibodies. But they can't confirm the presence of an autoimmune disease. Autoimmune diseases follow a waxing-and-waning disease path, meaning that symptoms will come and go over time as the underlying disease activity level changes. By the time a patient gets in to see their clinician, it is not uncommon that the symptoms have already abated.

The challenges posed by chronic diseases, while globally burdensome, are nearly universally exacerbated by flawed communication. In particular, those with, or at risk of, chronic disease would almost invariably achieve better outcomes if they were better empowered to communicate their health status in a timely manner, while health providers are disadvantaged by limited access to detailed, timely insight into patient health progressions that could dictate timely reactive and proactive measures.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Certain aspects of the present disclosure provide for an artificially intelligent method and system for prognostic evaluation of autoimmune disorders. In accordance with certain aspects of the present disclosure, the method and system may comprise one or more steps or system operations for presenting (e.g., with a conversational AI agent at one or more timepoints) a plurality of clinical conversational prompts to a patient user. The method and system may further comprise one or more steps or system operations for receiving (e.g., with the conversational AI agent at the one or more timepoints) a plurality of conversational responses from the patient user in response to the plurality of clinical conversational prompts. The method and system may further comprise one or more steps or system operations for receiving (e.g., with at least one server) a first set of text data comprising the plurality of conversational responses from the patient user. The method and system may further comprise one or more steps or system operations for receiving (e.g., with the at least one server via at least one application programming interface) a first set of medical record data for the patient user. In certain embodiments, the first set of medical record data for the patient user comprises at least one of blood biomarker test data and biometric measurement data. The method and system may further comprise one or more steps or system operations for processing (e.g., with the at least one server) the first set of text data and the first set of medical record data for the patient user according to a natural language processing model. In certain embodiments, the natural language processing model is configured to extract one or more features from the first set of text data and the first set of medical record data. In certain embodiments, the natural language processing model is configured to cluster one or more text segments from the first set of text data and the first set of medical record data according to the one or more features. The method and system may further comprise one or more steps or system operations for analyzing, according to the natural language processing model, the one or more text segments to determine one or more temporal or circumstantial association between the one or more text segments and the blood biomarker test data and/or the biometric measurement data. The method and system may further comprise one or more steps or system operations for assigning, according to at least one output of the natural language processing model, one or more prognostic values to the one or more text segments. The method and system may further comprise one or more steps or system operations for analyzing (e.g., with the at least one server) the one or more prognostic values for the one or more text segments to generate a prognostic assessment for at least one autoimmune disorder for the patient user. The method and system may further comprise one or more steps or system operations for providing (e.g., with the at least one server) the prognostic assessment to a practitioner user via a graphical user interface of a client device.

In accordance with certain aspects of the present disclosure, the method and system may further comprise one or more steps or system operations for configuring (e.g., with the at least one server) a conversational AI model according to the one or more prognostic values for the one or more text segments. In certain embodiments, the conversational AI model may comprise a large language model. In certain embodiments, the plurality of clinical conversational prompts may comprise a plurality of generative prompts according to the conversational AI model. In certain embodiments, the prognostic assessment comprises a diagnostic assessment for the at least one autoimmune disorder. In certain embodiments, the prognostic assessment comprises a predictive assessment of at least one pathophysiological event associated with the at least one autoimmune disorder. In certain embodiments, the prognostic assessment comprises a clinical recommendation for at least one pharmaceutical intervention for the patient user. In certain embodiments, the prognostic assessment comprises a clinical recommendation for at least one blood biomarker test for the patient user. In certain embodiments, the biometric measurement data comprises data from at least one body-worn sensor of the patient user. In accordance with certain aspects of the present disclosure, the method and system may further comprise one or more steps or system operations for modifying, via the conversational AI model, the plurality of clinical conversational prompts according to the one or more prognostic values for the one or more text segments.

Further aspects of the present disclosure provide for an artificially intelligent method and system for patient phenotyping for autoimmune disorders. In accordance with certain aspects of the present disclosure, the method and system may comprise one or more steps or system operations for presenting (e.g., with a conversational AI agent communicably engaged with a first server) a first set of clinical conversational prompts to a patient user according to a generative AI model. The method and system may comprise one or more steps or system operations for receiving (e.g., with the conversational AI agent) a first set of responses to the first set of clinical conversational prompts from the patient user. The method and system may comprise one or more steps or system operations for receiving (e.g., with the first server) a first set of medical record data for the patient, wherein the first set of medical record data comprises at least one of blood biomarker test data and biometric measurement data for the patient user. The method and system may comprise one or more steps or system operations for receiving (e.g., with at least one server) a first set of text data comprising the first set of responses to the first set of clinical conversational prompts from the patient user. The method and system may comprise one or more steps or system operations for processing (e.g., with the at least one server) the first set of text data and the first set of medical record data according to a natural language processing model. In accordance with certain aspects of the present disclosure, the natural language processing model is configured to extract one or more features from the first set of text data and the first set of medical record data. In accordance with said aspects, the natural language processing model is configured to cluster one or more text segments from the first set of text data and the first set of medical record data according to the one or more features. The method and system may comprise one or more steps or system operations for analyzing, according to the natural language processing model, the one or more text segments to determine one or more temporal or circumstantial association between the one or more text segments and the blood biomarker test data and/or the biometric measurement data. The method and system may comprise one or more steps or system operations for configuring, according to at least one output of the natural language processing model, a patient phenotype for the patient user according to the one or more temporal or circumstantial association between the one or more text segments and the blood biomarker test data and/or the biometric measurement data. In accordance with certain aspects of the present disclosure, the patient phenotype comprises one or more symptom, marker, and pathology trigger for an autoimmune disorder for the patient user. The method and system may comprise one or more steps or system operations for providing, with the at least one server, the patient phenotype for the patient user to a practitioner user via a graphical user interface of a client device.

In accordance with certain aspects of the present disclosure, the method and system may further comprise one or more steps or system operations for configuring (e.g., with the at least one server) the generative AI model according to the patient phenotype. The method and system may further comprise one or more steps or system operations for presenting (e.g., with the conversational AI agent) a second set of clinical conversational prompts to a patient user according to the generative AI model. In certain embodiments, the second set of clinical conversational prompts are configured according to the patient phenotype (e.g., per the generative AI model). In accordance with certain aspects of the present disclosure, at least one clinical prompt in the second set of clinical conversational prompts is different than the first set of clinical conversational prompts. The method and system may further comprise one or more steps or system operations for receiving (e.g., with the conversational AI agent) a second set of responses to the second set of clinical conversational prompts from the patient user. The method and system may further comprise one or more steps or system operations for receiving (e.g., with the first server) a second set of medical record data for the patient, wherein the second set of medical record data comprises a second set of blood biomarker test data and/or a second set of biometric measurement data for the patient user. The method and system may further comprise one or more steps or system operations for receiving (e.g., with the at least one server) a second set of text data comprising the second set of responses to the second set of clinical conversational prompts from the patient user. The method and system may further comprise one or more steps or system operations for processing (e.g., with the at least one server) the second set of text data according to the natural language processing model. In certain embodiments, the natural language processing model is configured to extract one or more features from the second set of text data according to the patient phenotype. In certain embodiments, the natural language processing model is configured to cluster one or more text segments from the second set of text data according to the one or more features. The method and system may further comprise one or more steps or system operations for processing (e.g., with the at least one server) the second set of text data and the second set of medical record data according to the natural language processing model. In accordance with certain aspects of the present disclosure, the natural language processing model is configured to extract one or more features from the second set of text data and the second set of medical record data according to the patient phenotype. In certain embodiments, the natural language processing model is configured to cluster one or more text segments from the second set of text data and the second set of medical record data according to the one or more features. The method and system may further comprise one or more steps or system operations for analyzing (e.g., according to a machine learning model) the one or more text segments to generate a prognostic assessment for the autoimmune disorder for the patient user. The method and system may further comprise one or more steps or system operations for providing (e.g., with the at least one server) the prognostic assessment to the patient user via a graphical user interface of an end user device associated with the patient user and/or the practitioner user via the graphical user interface of the client device. The method and system may further comprise one or more steps or system operations for updating or modifying the patient phenotype according to at least one output of the natural language processing model. In certain embodiments, the prognostic assessment comprises one or more recommended actions for management of the autoimmune disorder for the patient user.

Still further aspects of the present disclosure provide for an artificially intelligent method and system for identifying diagnostic triggers for an autoimmune disorder from clinical conversational data. In accordance with certain aspects of the present disclosure, the method and system may comprise one or more steps or system operations for presenting (e.g., with at least one server communicably engaged with a first client device) a plurality of clinical conversation prompts to a patient user at a user interface of the first client device. The method and system may further comprise one or more steps or system operations for receiving (e.g., with the at least one server via the first client device) a plurality of user-generated responses to the plurality of clinical conversation prompts from the patient user, the plurality of user-generated responses to the plurality of clinical conversation prompts comprising a first set of clinical conversation data. The method and system may further comprise one or more steps or system operations for receiving (e.g., with the at least one server) a first set of medical record data for the patient user, wherein the first set of medical record data comprises at least one of blood biomarker test data and biometric measurement data for the patient user. The method and system may further comprise one or more steps or system operations for processing (e.g., with the at least one server) the first set of clinical conversation data and the first set of medical record data according to a natural language processing model. In certain embodiments, the natural language processing model is configured to extract one or more features from the first set of clinical conversation data and the first set of medical record data. The natural language processing model may be configured to cluster one or more text segments from the first set of clinical conversation data and the first set of medical record data according to the one or more features. The method and system may further comprise one or more steps or system operations for analyzing, according to at least one output of the natural language processing model, the one or more text segments to determine one or more temporal or circumstantial association between the one or more text segments and the blood biomarker test data and/or the biometric measurement data. The method and system may further comprise one or more steps or system operations for configuring, according to the at least one output of the natural language processing model, one or more diagnostic triggers for the patient user according to the one or more temporal or circumstantial association between the one or more text segments and the blood biomarker test data and/or the biometric measurement data. The method and system may further comprise one or more steps or system operations for communicating (e.g., with the at least one server via a network interface) the one or more diagnostic triggers for the patient user to a practitioner user via a graphical user interface of a second client device.

In accordance with certain aspects of the present disclosure, the method and system may further comprise one or more steps or system operations for configuring (e.g., with the at least one server) the natural language processing model according to the one or more diagnostic triggers. In certain embodiments, the plurality of clinical conversation prompts are configured according to a generative AI model. In said embodiments, the generative AI model may comprise a large language model. The method and system may further comprise one or more steps or system operations for configuring (e.g., with the at least one server) the generative AI model according to the one or more diagnostic triggers. The method and system may further comprise one or more steps or system operations for presenting (e.g., with the at least one server communicably engaged with the first client device) a second or subsequent plurality of clinical conversation prompts to the patient user at the user interface of the first client device. The method and system may further comprise one or more steps or system operations for receiving (e.g., with the at least one server via the first client device) a second or subsequent plurality of user-generated responses to the second or subsequent plurality of clinical conversation prompts from the patient user, the second or subsequent plurality of user-generated responses to the second or subsequent plurality of clinical conversation prompts comprising a second or subsequent set of clinical conversation data. The method and system may further comprise one or more steps or system operations for processing (e.g., with the at least one server) the second or subsequent set of clinical conversation data according to the natural language processing model to extract one or more features from the second or subsequent set of clinical conversation data according to the one or more diagnostic triggers. The method and system may further comprise one or more steps or system operations for analyzing, according to a machine learning model, at least one output of the natural language processing model to identify at least one diagnostic trigger from the second or subsequent set of clinical conversation data. The method and system may further comprise one or more steps or system operations for communicating (e.g., with the at least one server) the at least one diagnostic trigger from the second or subsequent set of clinical conversation data to the practitioner user via the graphical user interface of the second client device. The method and system may further comprise one or more steps or system operations for communicating (e.g., with the at least one server) the at least one diagnostic trigger from the second or subsequent set of clinical conversation data to the patient user at the user interface of the first client device. The method and system may further comprise one or more steps or system operations for generating, according to the machine learning model, at least one clinical recommendation for management of the autoimmune disorder according to the at least one diagnostic trigger from the second or subsequent plurality of user-generated responses. The method and system may further comprise one or more steps or system operations for communicating (e.g., with the at least one server) the at least one clinical recommendation for management of the autoimmune disorder to the practitioner user via the graphical user interface of the second client device. The method and system may further comprise one or more steps or system operations for communicating (e.g., with the at least one server) the at least one clinical recommendation for management of the autoimmune disorder to the patient user at the user interface of the first client device.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system, method and computer-program product of the present disclosure may be better understood from the following illustrative description with reference to the following drawings in which:

FIG. 1 is an illustrative embodiment of a computing system through which one or more aspects of the present disclosure may be implemented;

FIG. 2 is an architecture diagram of an artificially intelligent system for prognostic evaluation of autoimmune disorders, in accordance with certain aspects of the present disclosure;

FIG. 3 is a flow diagram of an artificially intelligent system and method for prognostic evaluation of autoimmune disorders, in accordance with certain aspects of the present disclosure;

FIG. 4 is a functional block diagram of an artificially intelligent system and method for prognostic evaluation of autoimmune disorders, in accordance with certain aspects of the present disclosure;

FIG. 5 is a process flow diagram of an artificially intelligent system and method for prognostic evaluation of autoimmune disorders, in accordance with certain aspects of the present disclosure;

FIG. 6 is a process flow diagram of a routine for an artificially intelligent system and method for prognostic evaluation of autoimmune disorders, in accordance with certain aspects of the present disclosure;

FIG. 7 is a process flow diagram of a routine for an artificially intelligent system and method for prognostic evaluation of autoimmune disorders, in accordance with certain aspects of the present disclosure;

FIG. 8 is a process flow diagram of a routine for an artificially intelligent system and method for prognostic evaluation of autoimmune disorders, in accordance with certain aspects of the present disclosure;

FIG. 9 is a process flow diagram of an artificially intelligent method for prognostic evaluation of autoimmune disorders, in accordance with certain aspects of the present disclosure;

FIG. 10 is a process flow diagram of an artificially intelligent method for prognostic evaluation of autoimmune disorders, in accordance with certain aspects of the present disclosure; and

FIG. 11 is a process flow diagram of an artificially intelligent method for prognostic evaluation of autoimmune disorders, in accordance with certain aspects of the present disclosure.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatuses and systems configured to facilitate the acquisition, management and practical application of health information arising from a) medical records, b) biometric profiling, and c) medical communications for people (users or patients) whose health status suggests that they would benefit from proactive health monitoring and healthcare facilitation.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Before the present invention and specific exemplary embodiments of the invention are described, it is to be understood that this invention is not limited to the particular embodiments described, and as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, “exemplary” means serving as an example or illustration and does not necessarily denote ideal or best.

As used herein, the terms “computer,” “processor” and “computer processor” encompass a personal computer, a workstation computer, a tablet computer, a smart phone, a microcontroller, a microprocessor, a field programmable object array (FPOA), a digital signal processor (DSP), an application-specific integrated circuit (ASIC), a field programmable gate array (FPGA), a programmable logic array (PLA), or any other digital processing engine, device or equivalent capable of executing software code including related memory devices, transmission devices, pointing devices, input/output devices, displays and equivalents.

As used herein, the terms “conversational agent” or “conversational AI agent” or “agent” refer to any device, system and/or program configured to autonomously execute one or more objective function in response to one or more inputs. Said terms may be used interchangeably. The one or more inputs may comprise one or more user-generated inputs, sensor-based inputs, internal system inputs, external system inputs, environmental percepts, and the like. Examples of conversational agents may include, but are not limited to, one or more virtual assistant, personal assistant or chatbot.

As used herein, the term “mobile device” includes any portable electronic device capable of executing one or more digital functions or operations; including, but not limited to, smart phones, tablet computers, personal digital assistants, wearable activity trackers, smart watches, smart speakers, and the like.

As used herein, the terms “provider” and “practitioner” refer to a healthcare professional or healthcare provider that is responsible for one or more aspects of a patient's care; including, but not limited to, a doctor, a nurse, a physician's assistant, a pharmacist, a technician, and the like. The terms “provider” and “practitioner” may be used interchangeably throughout the present disclosure. As used herein, the term “practitioner user” refers to a provider/practitioner who is also a user of the systems and methods described herein.

As used herein, the term “patient” refers to any recipient of health care services that are performed or facilitated by a practitioner; including, but not limited to, an individual with an autoimmune disorder. As used herein, the term “patient user” refers to a patient who is also a user of the systems and methods described herein.

As used herein, the term “includes” means includes but is not limited to, the term “including” means including but not limited to. The term “based on” means based at least in part on.

As used herein, the term “interface” refers to any shared boundary across which two or more separate components of a computer system may exchange information. The exchange can be between software, computer hardware, peripheral devices, humans, and combinations thereof.

As used herein, the term “transmit” or “communicate” and their conjugates means transmission of digital and/or analog signal information by electronic transmission, Wi-Fi, BLUETOOTH technology, wireless, wired, or other known transmission technologies including transmission to an Internet web site.

As used herein, the term “biometric” refers to any measurable biological (i.e., anatomical and/or physiological) and/or behavioral characteristic of a human person (i.e., a patient). In accordance with certain aspects of the present disclosure, examples of biometric measurements may include, but are not limited to, heart rate and heart activity (e.g., pulse, and electrocardiogram), sleep data, brain wave data (e.g., MRI and fMRI), activity data (i.e., movement/telemetry), body temperature, blood pressure, and the like.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this embodiment, one or more computer programs that when executed perform methods of the present technology need not reside on a single computer or processor but may be distributed in a modular fashion amongst a number of different computers or processors to implement various aspects of the present technology. Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Certain benefits and advantages of the present disclosure include an artificially intelligent method and system configured to identify and train diagnostically relevant triggers to alert medical providers regarding emergent pathology risks or pharmaceutical intervention opportunities in the treatment and management of autoimmune diseases.

Certain objects and advantages of the present disclosure include artificially intelligent methods and system for analyzing (e.g., according to one or more machine learning framework) written and oral content of interpersonal communications that contain subjective information about the state of a person's health. Embodiments of the present disclosure include methods and systems for analytically tractable acquisition and management of clinical communication data for diagnostic and prognostic purposes. Embodiments of the present disclosure include methods and systems for systematically leveraging incumbent information in concert with rigorously quantifiable medical data, such as laboratory tests and other standardized diagnostics.

Certain objects and advantages of the present disclosure include artificially intelligent methods and systems for effective integration of communication-related insights into diagnostic and prognostic digital health resources. Exemplary systems, methods, and apparatuses according to the principles herein may comprise machine learning and deep learning techniques for the development of one or more quantitative metrics derived from clinical conversational data. Such techniques may include natural language processing (NLP) and generative artificial intelligence (gAI). In accordance with certain aspects of the present disclosure, NLP may comprise one or more computer-implemented machine learning framework for the analytical deconvolution of free text into informative granules and broad trends. In accordance with certain aspects of the present disclosure, gAI may comprise one or more artificial neural network comprising the capacity to simulate normal human communication. In accordance with certain aspects of the present disclosure, the gAI comprises a clinical conversational framework configured as an artificially intelligent vehicle by which to facilitate productive engagement between medical patients and digital health resources intended for improving the reactive and proactive healthcare for those patients.

Certain objects and advantages of the present disclosure include methods and systems configured to aggregate and analyze clinical conversational data, patient biometric data, medical records data blood biomarker test data, and patient biometric data to tune metrics that are increasingly specific to a patient in the creation of a dynamic patient phenotype to enhance clinical understanding of one or more factors that are specific to the patient's health status. In accordance with certain aspects of the present disclosure, the patient phenotype is used in the configuration and/or modification of one or more gAI models to drive one or more gAI communications tailored to the patient.

Certain objects and advantages of the present disclosure include one or more systems, methods, apparatuses, and digital platform products comprising one or more NLP and gAI engine and framework for presenting patients with a desirable (i.e., useful and not onerous) communication resource that fosters regular information exchanges that do not overly tax health providers, while also encouraging inquiries that are patient-specific and circumstantially adaptive in the way a person might share with someone that they know and trust. In accordance with certain embodiments, data derived from these information exchanges is communicated (e.g., in accordance with one or more communication protocols) to alert healthcare providers to the need to respond to medically-specific questions or requests from the patient, as well as scenarios where NLP-driven AI models detect a possible confluence of circumstances (e.g., potentially a set of concerning phrases in the transcript, in combination with specific recent test results) that may flag possible adverse events and circumstances indicating the need for one or more specific therapeutic interventions.

Certain objects and advantages of the present disclosure include one or more systems, methods, apparatuses, and digital platform products that facilitate proactive assessment and care of people with assessed risk of autoimmune diseases, including chronic connective tissue disorders. While the present disclosure discusses autoimmune diseases with some degree of specificity, the present systems, methods, apparatuses, and digital platform products may be further adaptable to numerous other conditions, diseases and disorders.

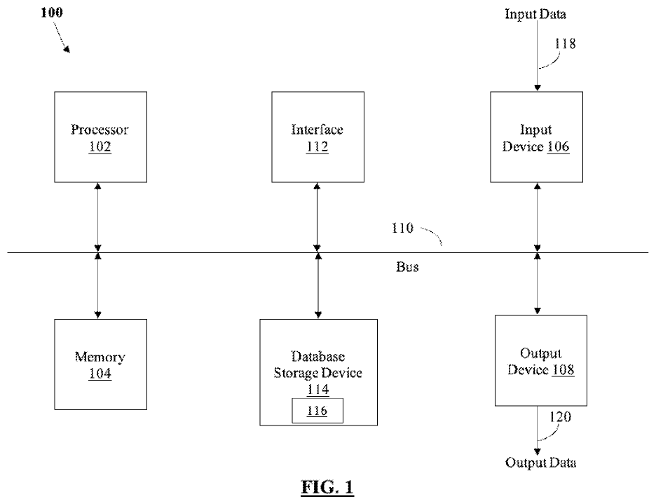

Turning now descriptively to the drawings, in which similar reference characters denote similar elements throughout the several views, FIG. 1 depicts an exemplary computing system in which certain illustrated embodiments of the present invention may be implemented.

Referring now to FIG. 1, a processor-implemented computing device in which one or more aspects of the present disclosure may be implemented is shown. According to an embodiment, a processing system 100 may generally comprise at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling the processing system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could comprise more than one distinct processing device, for example to handle different functions within the processing system 100. Input device 106 receives input data 118 and can comprise, for example, a keyboard, a pointer device such as a pen-like device or a mouse, audio receiving device for voice-controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can comprise, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, at least one database 116. The interface 112 may allow wired and/or wireless communication between the processing unit 102 and peripheral components that may serve a specialized purpose. In general, the processor 102 can receive instructions as input data 118 via input device 106 and can display processed results or other output to a user by utilizing output device 108. More than one input device 106 and/or output device 108 can be provided. It should be appreciated that the processing system 100 may be any form of terminal, server, specialized hardware, or the like.

It is to be appreciated that the processing system 100 may be a part of a networked communications system. Processing system 100 could connect to a network, for example the Internet or a WAN. Input data 118 and output data 120 could be communicated to other devices via the network. The transfer of information and/or data over the network can be achieved using wired communications means or wireless communications means. A server can facilitate the transfer of data between the network and one or more databases. A server and one or more databases provide an example of an information source.

Thus, the processing computing system environment 100 illustrated in FIG. 1 may operate in a networked environment using logical connections to one or more remote computers. The remote computer may be a personal computer, a server, a router, a network PC, a peer device, or other common network node, and typically includes many or all of the elements described above.

It is to be further appreciated that the logical connections depicted in FIG. 1 include a local area network (LAN) and a wide area network (WAN) but may also include other networks such as a personal area network (PAN). Such networking environments are commonplace in offices, enterprise-wide computer networks, intranets, and the Internet. For instance, when used in a LAN networking environment, the computing system environment 100 is connected to the LAN through a network interface or adapter. When used in a WAN networking environment, the computing system environment typically includes a modem or other means for establishing communications over the WAN, such as the Internet. The modem, which may be internal or external, may be connected to a system bus via a user input interface, or via another appropriate mechanism. In a networked environment, program modules depicted relative to the computing system environment 100, or portions thereof, may be stored in a remote memory storage device. It is to be appreciated that the illustrated network connections of FIG. 1 are exemplary and other means of establishing a communications link between multiple computers may be used.

FIG. 1 is intended to provide a brief, general description of an illustrative and/or suitable exemplary environment in which embodiments of the below described present invention may be implemented. FIG. 1 is an example of a suitable environment and is not intended to suggest any limitation as to the structure, scope of use, or functionality of an embodiment of the present invention. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

In the description that follows, certain embodiments may be described with reference to acts and symbolic representations of operations that are performed by one or more computing devices, such as the computing system environment 100 of FIG. 1. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of well-known computing systems, environments, and configurations that may be suitable for use with an embodiment include, but are not limited to, personal computers, handheld or laptop devices, personal digital assistants, multiprocessor systems, microprocessor-based systems, set top boxes, programmable consumer electronics, network, minicomputers, server computers, game server computers, web server computers, mainframe computers, and distributed computing environments that include any of the above systems or devices.

Embodiments may be described in a general context of computer-executable instructions, such as program modules, being executed by a computer. Generally, program modules include routines, programs, objects, components, data structures, etc., that perform particular tasks or implement particular abstract data types. An embodiment may also be practiced in a distributed computing environment where tasks are performed by remote processing devices that are linked through a communications network. In a distributed computing environment, program modules may be located in both local and remote computer storage media including memory storage devices.

With the exemplary computing system environment 100 of FIG. 1 being generally shown and discussed above, description will now turn towards illustrated embodiments of the present invention which generally relates to systems and methods for presentation and analysis of clinical conversation data to assist in prognostic evaluation and personalized management of autoimmune diseases.

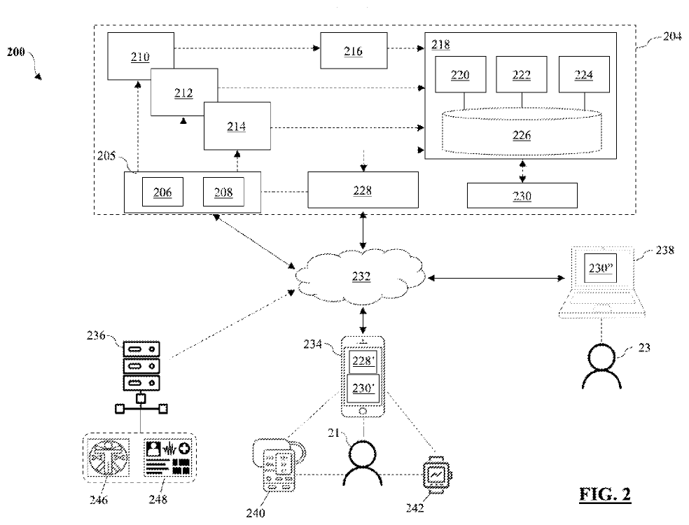

Referring now to FIG. 2, an architecture diagram of an artificially intelligent system 200 for prognostic evaluation of autoimmune disorders is shown. In accordance with certain aspects of the present disclosure, system 200 comprises a computing architecture 204 comprising an artificial intelligence analytical framework for acquiring, managing, and progressively learning from information and associative relationships in patient profiles, communications and medical records. In accordance with certain embodiments, system 200 may comprise a patient client device 234, a practitioner client device 238, and one or more electronic health records (EHR) server 236 communicably engaged with one or more components of computing architecture 204 via a network communications interface 232. In accordance with certain aspects of the present disclosure, system 200 enables one or more practitioner user 23 to proactively evaluate a variety of datapoints to improve the time to diagnose and personalized management of one or more autoimmune disease of a patient user 21. The one or more autoimmune disease may include, but are not limited to, SLE, rheumatoid arthritis, scleroderma, polymyositis, Sjogren's syndrome, Raynaud's syndrome and Mixed Connective Tissue Disease. In accordance with certain aspects of the present disclosure, patient client device 234 may facilitate an exchange of patient-specific data to and from computing architecture 204 via network communications interface 232. Patient client device 234 may be communicably engaged with one or more of a physiological measurement device 240 and a body-worn sensor device 242 via a wireless (e.g., BLUETOOTH) or wireline (e.g., USB) interface. Physiological measurement device 240 and body-worn sensor device 242 may be configured to collect a plurality of biometric data from patient user 21. In accordance with certain aspects of the present disclosure, the biometric data may comprise heart rate data, heart rate variability data, ECG data, sleep data, activity (i.e., movement/telemetry) data, blood pressure, temperature, pulse oximetry, and the like. In certain embodiments, physiological measurement device 240 and body-worn sensor device 242 may be communicably engaged directly with computing architecture 204 via network communications interface 232 (e.g., without a communications interface with patient client device 234). In accordance with certain embodiments, patient client device 234 may send biometric data from physiological measurement device 240 and body-worn sensor device 242 to practitioner client device 238 (e.g., according to one or more data transfer protocols) via network communications interface 232. In accordance with certain aspects of the present disclosure, EHR server 236 may comprise electronic health record (EHR) data 248 and/or laboratory test data 246 for patient user 21 stored thereon. EHR data 248 may comprise a plurality of longitudinal health data for patient user 21, as well as a plurality of EHR data specific to the diagnosis, management and treatment of one or more autoimmune condition for user 21. Laboratory test data 246 may comprise blood biomarker test data related to diagnosis, management and treatment of one or more autoimmune condition for user 21. EHR server 236 may communicate (e.g., according to one or more data transfer protocols) EHR data 248 and/or laboratory test data 246 to computing architecture 204 via network communications interface 232. In accordance with certain embodiments, EHR server 236 may send and receive EHR data 248 and/or laboratory test data 246 to and from practitioner client device 238 (e.g., according to one or more data transfer protocols) via network communications interface 232.

In accordance with certain aspects of the present disclosure, computing architecture 204 may comprise an application programming interface (API) gateway 205 configured to facilitate data transfer between computing architecture 204 and one or more other elements of system 200. In certain embodiments, API gateway 205 may comprise a lambda function 206 and an integration service 208. Lambda function 206 comprises an event-driven function for managing computing resources for computing architecture 204. In various embodiments, lambda function 206 (or an equivalent function) is configured to scale up a run-time environment, execute one or more functions (e.g., handling medical record uploads), and scale the run-time function down as necessary to efficiently manage computing resources. An example of lambda function 206 includes AWS LAMBDA, available from AMAZON WEB SERVICES, Seattle, WA. Integration service 208 may comprise a bi-directional data transfer interface for managing the flow of data to and from computing architecture 204 and one or more elements of system 200. An example of integration service 208 may include AMAZON APPFLOW, available from AMAZON WEB SERVICES, Seattle, WA. In accordance with certain aspects of the present disclosure, computing architecture 204 may further comprise one or more webservice functions 210-214. In certain embodiments, a first webservice function 210 may comprise a simple storage service function configured to provide object storage for computing architecture 204. First webservice function 210 may comprise a bucket-type storage architecture and may be configured to manage the storage and routing of a plurality of object files (e.g., medical records data and laboratory test data). An example of first webservice function 210 may include AMAZON S3, available from AMAZON WEB SERVICES, Seattle, WA. In certain embodiments, a second webservice function 212 may comprise a real-time data handling function. Second webservice function 212 may comprise a scalable and durable real-time data streaming service that captures and processes data from multiple sources in real-time. Second webservice function 212 may facilitate a real-time data transfer interface between patient client device 234, physiological measurement device 240 and body-worn sensor device 242, among others. An example of second webservice function 212 may include AMAZON KINESIS, available from AMAZON WEB SERVICES, Seattle, WA. In certain embodiments, a third webservice function 214 may comprise a relational database function for setup, operation, and scaling of one or more relational database for use in a care management application 230 (as described in more detail below). An example of a third webservice function 214 may include AMAZON RDS, available from AMAZON WEB SERVICES, Seattle, WA.

In accordance with certain aspects of the present disclosure, computing architecture 204 may comprise an optical character recognition (OCR) engine 216. OCR engine 216 may be communicably engaged with first webservice function 210 to receive one or more medical records files or laboratory test data files. The one or more medical records files or laboratory test data files may comprise scanned PDF document and/or image file formats. OCR engine 216 may be operably configured to process the one or more medical records files or laboratory test data files according to a plurality of pattern-matching algorithms to enable data extraction from printed or written text from a scanned document or image file. OCR engine 216 may be operably configured to convert the text into a machine-readable form to be used for further data processing (e.g., by AI engine 218). In accordance with certain aspects of the present disclosure, computing architecture 204 may comprise an artificial intelligence (AI) engine 218. AI engine 218 may be communicably engaged to receive a plurality of data streams/inputs from first webservice function 210, second webservice function 212, third webservice function 214 and/or OCR engine 216. AI engine 218 may comprise one or more AI frameworks (i.e., models) for acquiring, managing, and progressively learning from information and associative relationships in patient profiles, communications and medical records in accordance with one or more aspects of system 200. In accordance with certain embodiments, AI engine 218 may comprise one or more sub-engines, including a generative AI (gAI) engine 220, a natural language processing (NLP) engine 222, and a machine learning (ML) engine 224. In accordance with certain aspects of the present disclosure, gAI engine 220 comprises a neural network architecture configured to identify the patterns and structures within existing data to generate new and original content (e.g., clinical conversational interactions). In certain embodiments, gAI engine 220 comprises a large language model. In certain embodiments, gAI engine 220 is configured to generate clinical conversational content to facilitate multi-turn conversational interactions between a conversational AI agent 228 and patient user 21. In accordance with certain aspects of the present disclosure, gAI engine 220 encompasses a class of computational techniques that aim to realistically simulate human expression (e.g., communication) by assimilating a given circumstance (e.g., a communication prompt, such as, “I felt dizzy this morning getting out of bed. Should I be concerned?”) with a prescribed context (e.g., the response should be commensurate with that of a medical professional trained in immunometabolic disorders), and should abide by specific semantic rules (e.g., the response should be friendly and solicitous, and should seek supporting information, while avoiding aggressive or invasive interrogative behavior). The manner in which such simulated behavior is trained entails the application of deep learning algorithms to process a volume of previously compiled examples of human expression that address both the context (i.e., a body of formal and informal literature in which numerous examples of terminology and concepts of immunometabolic physiology and pathology are conveyed) and semantics (i.e., specific examples of effective communication and inquiry to be emulated, and other examples of undesirable communication to be avoided). In accordance with certain aspects of the present disclosure, gAI engine 220 comprises a gAI model that is rigorously trained (by exposure to examples of best-practice medical communication) to encourage patient input and slant it toward a normal “bedside manner” mode of interaction with patient user 21 that mixes medically-relevant topics with non-technical “chatting,” where the latter may provide extra-topical clues as to the mood or health of the participant/patient. In accordance with certain aspects of the present disclosure, the gAI model is configured to steer conversations toward assessing specific medically relevant queries, while avoiding any suggestive bias toward any specific answer.

In accordance with certain aspects of the present disclosure, NLP engine 222 comprises one or more NLP model comprising one or more NLP algorithms configured to process text data derived from medical record data (e.g., received from OCR engine 216) and patient conversational data (e.g., in response to a plurality of generative outputs from gAi engine 220) to derive one or more subsets of data that is of direct (or statistically probable) relevance to criteria that inform a disease-relevant health status of patient user 21. In accordance with certain embodiments, NLP engine 222 comprises one or more NLP model comprising one or more NLP algorithms configured to parse text data such that textual granules (phrases, sentences, paragraphs, etc.) can be targeted for extraction according to contextually sensitive criteria (e.g., an interest in “expression,” but in the specific context of elevated or suppressed expression of a given protein marker, as opposed to all other contexts in which the word may be used). In accordance with certain embodiments, NLP engine 222 comprises one or more NLP model comprising one or more NLP algorithms configured to analyze the text data to generate one or more quantitative metrics, where quantification may assess the functional similarity to the original query, the temporal or spatial proximity within a document to other queries, or corresponding sentiment context to suggest, for example, to assign a prognostic value to a given term or terms for patient user 21. Such quantification supports a systematic parsing of text documents so that granules of textual insight may be grouped temporally or circumstantially with other forms of data, such as test results or biometric measurements. Such groupings enable the formulation of associations, so that text granules frequently observed at times closely preceding or proximal to key medical instances (e.g., flares, relapses, emerging diagnoses, etc.) may be ascribed elevated prognostic value for future monitoring and clinical management of patient 21.

In accordance with certain aspects of the present disclosure, ML engine 224 comprises one or more ML model comprising one or more ML algorithms configured to process MR and conversational data and one or more outputs from NLP engine 222 to generate one or more clinical recommendation, clinical insights, pharmaceutical interventions, laboratory test recommendation, and/or facilitation of one or more interactions between patient user 21 and provider user 23. Exemplary ML models that may be incorporated into ML engine 224 may include, but are not limited, deep learning models, such as the deep Boltzmann machine, deep belief networks, Recurrent Neural Network (RNN), Fully Convolutional Neural Network (FCN), Dilated Residual Network (DRN), Generative Adversarial Network (GAN), and Deep Neural Network (DNN); ensemble, such as random forest, gradient boost machines, boosting, adaboosting, stacked generalization, and gradient boosted regression trees; neural networks, such as perception, back-propagation, Hopfield, ridge regression, LASSO, and elastic; rule systems, such as cubist, one rule, and zero rule; linear regression, such as ordinary least squares regression, stepwise regression, multivariate adaptive regression splines, locally estimated scatterplot smoothing, and logistic regression; Bayesian, such as naïve, averaged one-dependent estimators, Gaussian naïve, and multinomial naïve; decision tree, such as classification and regression, iterative dichotomiser, and condition-decision; instance-based, such k-nearest neighbor, learning vector quantization, and locally weighted learning; and clustering, such as k-means, k-medians, expectation max, and hierarchical.

In accordance with certain aspects of the present disclosure, computing architecture 204 may further comprise an application database 226 communicably engaged with AI engine 218 to facilitate data transfer and storage between one or more of first webservice function 210, second webservice function 212, third webservice function 214, and OCR engine 216. Application database 226 may store one or more model configurations for, and/or model outputs from, gAI engine 220, NLP engine 222 and ML engine 224. In accordance with certain embodiments, AI engine 218 may be communicably engaged with a conversational AI agent 228 configured to present one or more generative clinical conversational prompts to patient user 21 and facilitate one or more multi-turn conversational interactions with patient user 21 (e.g., via patient user instance 228′). Patient user instance 228′ of conversational AI agent 228 may be configured to present the generative clinical conversational prompts to patient user 21 via a user interface of patient user device 234 and may be configured to receive a plurality of patient responses to the generative clinical conversational prompts via at least one input device of patient user device 234. Computing architecture 204 may further comprise a care management application 230. Care management application 230 may comprise a plurality of application modules, functions and processor-executable operations for personalized management of one or more autoimmune disease of patient user 21. In accordance with certain aspects of the present disclosure, care management application 230 may be operably engaged with AI engine 218 to provide one or more diagnostic insight clinical recommendation, clinical insights, pharmaceutical interventions, laboratory test recommendation to patient user 21 (e.g., via a patient user application instance 230′) and provider user 23 (e.g., via a practitioner user application instance 230″) and/or facilitate one or more communications or real-time interactions between patient user 21 and provider user 23.

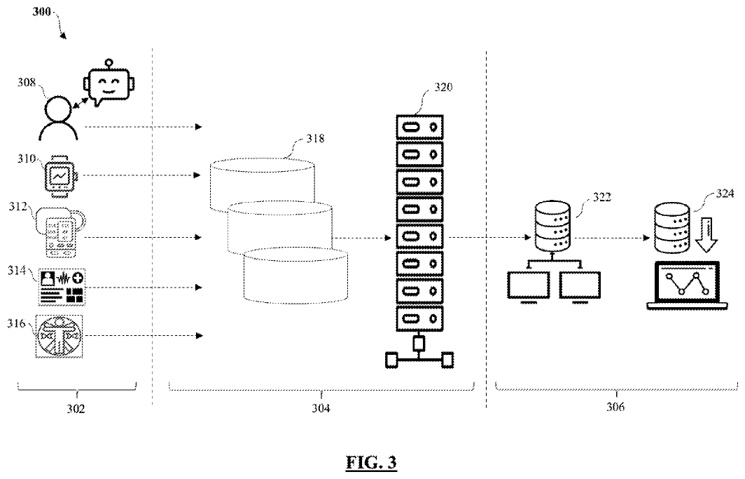

Referring now to FIG. 3, a flow diagram of an artificially intelligent system 300 for prognostic evaluation of autoimmune disorders is shown. In accordance with certain aspects of the present disclosure, system 300 is equivalent to system 200, as shown and described in FIG. 2. In accordance with certain aspects of the present disclosure, system 300 illustrates a flow of data and associated operations from data sources 302, to an application computing environment 304, to client interfaces 306. In accordance with certain embodiments, data sources 302 comprise a conversational AI agent 308 (i.e., a chatbot), a wearable sensor device 310, a physiological sensor device 312, patient medical record data 314, and laboratory testing data 316. System 300 is configured to aggregate multiple data types across data sources 302 in order to receive and process a plurality of patient-specific data within application computing environment 304. The patient-specific data includes patient-reported data from various applications, sensor-based information from wearable devices, medical records, survey and questionnaire data, as well as other external data. In accordance with certain aspects of the present disclosure, application computing environment 304 is configured to receive and process the data via webservices 318. Webservices 318 may handle the data and provide the data to application server 320. Application server 320 may comprise one or more gAI engine, Natural Language Processing engine, and machine learning engine (e.g., deep learning engine) to analyze data and set it on an iterative learning and refinement mode. The Natural Language Processing engine, gAI engine, and machine learning engine are configured to analyze the data to generate intelligent recommendations for patient identification, treatment and other interventions, and track outcomes. The recommendations for patient identification, treatment and other interventions may be delivered to one or more client devices 322. Patient outcome data and other patient management data may be presented at one or more graphical user interfaces 324.

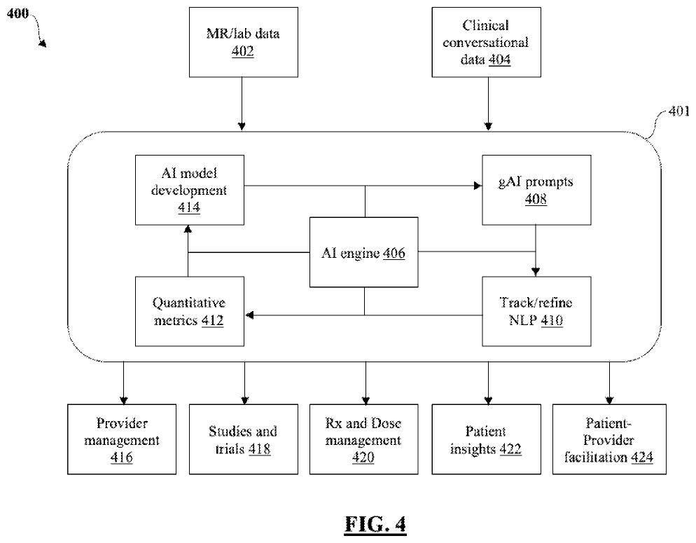

Referring now to FIG. 4, a functional block diagram 400 of an artificially intelligent system and method for prognostic evaluation of autoimmune disorders is shown. In accordance with certain aspects of the present disclosure, the artificially intelligent system for prognostic evaluation of autoimmune disorders comprises system 200, as shown and described in FIG. 2. In accordance with certain aspects of the present disclosure, system computing modules 401 may be configured to receive/ingest a plurality of data inputs comprising medical record and laboratory test data 402 and clinical conversation data 404. In accordance with certain aspects of the present disclosure, system computing modules 401 may be executed within computing architecture 204, as shown and described in FIG. 2. In accordance with certain aspects of the present disclosure, system computing modules 401 may be configured to receive and process medical record and laboratory test data 402 and clinical conversation data 404 at AI engine 406. AI engine 406 may be configured to execute one or more gAI model, NLP model and ML model in accordance with blocks 408-414. In accordance with certain embodiments, system 400 may be configured to generate a plurality of gAI prompts (Block 408) in accordance with a gAI model of AI engine 406. The plurality of gAI prompts may drive a plurality of multi-turn conversational interactions with a patient user to derive the clinical conversation data 404 via an AI conversational agent. The clinical conversation data 404 may be processed in accordance with an NLP model (Block 410) to identify one or more temporal and/or circumstantial associations between the medical record and laboratory test data 402 and clinical conversation data 404. The NLP model may be continuously refined (Block 410) according to the one or more temporal and/or circumstantial associations between the medical record and laboratory test data 402 and clinical conversation data 404. One or more outputs of the NLP model may be analyzed by at least machine learning model or deep learning model to derive one or more quantitative metrics for deriving one or more clinical recommendation, clinical insights, pharmaceutical interventions, laboratory test recommendation, and/or to facilitation of one or more patient-practitioner interactions. An output of Block 410 may be used to drive a feedback loop at Block 414 to further refine/develop one or more AI model (e.g., gAI model, NLP model and/or ML model.

In accordance with certain aspects of the present disclosure, system 400 fosters adaptive refinement, such that a growing body of disease-specific information (e.g., medical records, test results, biometric observations and key textual phrases, concepts and relationships therein) can be periodically subjected to feature selection analysis in order to identify specific informational features or combinations thereof that tend to coincide with, or precede, important changes in patient status. This permits the AI model(s) incumbent within system 400 to progressively refine, which sharpens both their specificity and accuracy in characterizing patient status and flagging emerging situations that may require medical intervention. Additionally, a set of sham metrics (measurements, observations or textual instances that are not known to be disease-relevant) can be maintained in parallel, and periodically explored using feature selection in order to potentially determine, de novo, whether previously unidentified features should be monitored for potential disease-relevance. This fosters pathophysiological and pharmaceutical learning as a byproduct of continued monitoring.

In accordance with certain aspects of the present disclosure, system computing modules 401 may drive one or more system outputs 416-424. Said system outputs may enable a plurality of practical application functions for one or more stakeholder users of a care management application for personalized management of autoimmune conditions for a patient. In accordance with certain embodiments, an output of system computing modules 401 may comprise a plurality of provider management outputs 416 comprising one or more clinical recommendation, clinical insights, pharmaceutical interventions, laboratory test recommendation for a provider user of system 400. In accordance with certain embodiments, an output of system computing modules 401 may comprise clinical study/trial outputs 418 including, for example, one or more recommendation for participant recruitment/enrollment based on the analysis of the medical record and laboratory test data 402 and clinical conversation data 404. In accordance with certain embodiments, an output of system computing modules 401 may comprise prescription and dose management outputs 420 including, for example, a recommendation for a pharmaceutical intervention or prescription titration to the patient user and/or the provider user based on the analysis of the medical record and laboratory test data 402 and clinical conversation data 404. In accordance with certain embodiments, an output of system computing modules 401 may comprise a plurality of patient insights 422 comprising one or more activity or behavioral recommendation, patient phenotype, pharmaceutical recommendation, laboratory test recommendation and pathophysiological insight for a patient user of system 400. In accordance with certain embodiments, an output of system computing modules 401 may comprise facilitation of one or more patient-provider interaction 424; for example, automated scheduling of a virtual or in-person medical appointment, phone call, email, text message or other communication.

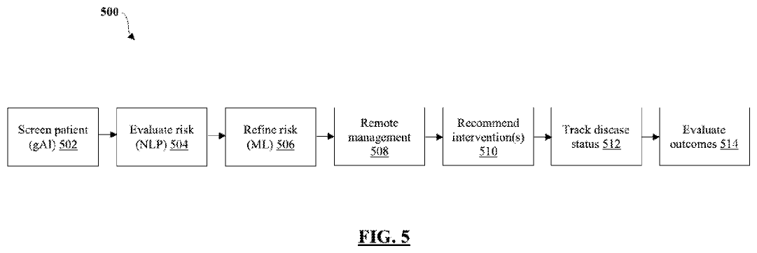

Referring now to FIG. 5, a diagram of a process flow 500 of an artificially intelligent system and method for prognostic evaluation of autoimmune disorders is shown. The operations of process flow 500 may be performed in the order presented, in a different order, or simultaneously. Further, in some exemplary embodiments, some of the operations may be omitted, added, modified, skipped, or the like without departing from the scope of the invention. In accordance with certain aspects of the present disclosure, the operations of process flow 500 may be embodied as one or more system routines of system 200, as shown and described in FIG. 2. In accordance with certain aspects of the present disclosure, process flow 500 may comprise one or more steps or operations for screening a patient according to a plurality of generative prompts generated by a generative AI model (Step 502). Step 502 may comprise a plurality of multi-turn interactions between a conversational AI agent (e.g., a chatbot) and the patient. The conversational AI agent may be configured to receive (e.g., by voice or text) a plurality of patient-generated responses to the plurality of generative prompts. Process flow 500 may proceed by performing one or more steps or operations for receiving and processing the plurality of patient-generated responses according to a natural language processing model to evaluate a degree of risk that the patient is exhibiting one or more symptoms or indications of an autoimmune disease (Step 504). Process flow 500 may further comprise one or more steps or operations for analyzing the plurality of patient-generated responses and/or one or more outputs of the natural language processing model according to at least one machine learning or deep learning framework to refine a level of risk for the patient and/or provide a recommended diagnosis for the patient (Step 506). Process flow 500 may further comprise one or more steps or operations for providing one or more remote management tools to the patient for management of the diagnosed autoimmune disease (Step 508). Step 508 may comprise one or more steps or operations for continuous data collection via one or more modalities, including sensor data, biometric data, clinical conversational data (e.g., according to the gAI model), blood biomarker test data, medical records data, and the like. Process flow 500 may further comprise one or more steps or operations for providing one or more recommended intervention(s) for management of the diagnosed autoimmune disease to the patient and/or one or more practitioner or stakeholder users (Step 510). Process flow 500 may further comprise one or more steps or operations for continuous monitoring and processing of patient data to track a disease status (e.g., progression, improvement, etc.) for the patient (Step 512). Process flow 500 may further comprise one or more steps or operations for analyzing system data, either ad hoc or according to one or more predefined intervals or milestones, to evaluate one or more clinical outcomes for the patient (Step 514).

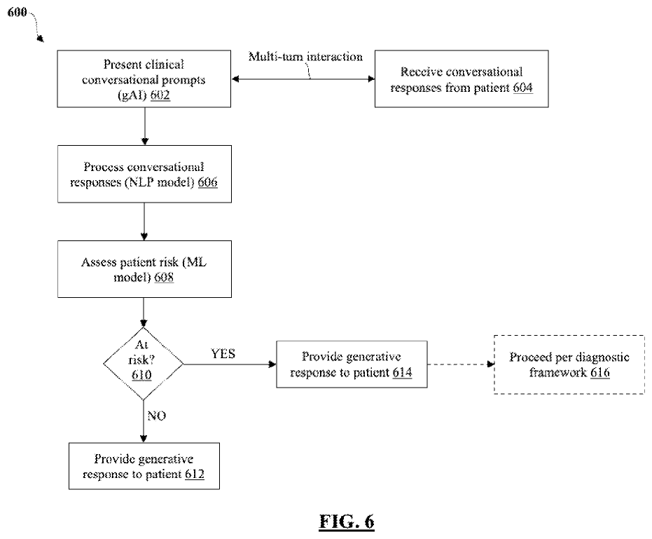

Referring now to FIG. 6, a process flow diagram of a routine 600 for an artificially intelligent system and method for prognostic evaluation of autoimmune disorders is shown. The operations of routine 600 may be performed in the order presented, in a different order, or simultaneously. Further, in some exemplary embodiments, some of the operations may be omitted, added, modified, skipped, or the like without departing from the scope of the invention. In accordance with certain aspects of the present disclosure, routine 600 may be embodied within one or more routines or operations of system 200, as shown and described in FIG. 2. Routine 600 may facilitate one or more steps of process flow 500, as shown in FIG. 5. In accordance with certain aspects of the present disclosure, routine 600 may comprise one or more steps or operations 602-616 for assessing a risk of an undiagnosed autoimmune condition in a patient user.

In accordance with certain aspects of the present disclosure, routine 600 may comprise one or more steps or operations for presenting a plurality of clinical conversational prompts to a patient user (e.g., according to a generative AI model) via a conversational AI agent (Step 602) and receiving a plurality of conversational responses from the patient user via the conversational AI agent (Step 604). In certain embodiments, steps 602-604 comprise a plurality of multi-turn (i.e., conversational) interactions between the patient user and the conversational AI agent. Routine 600 may proceed by executing one or more steps or operations for processing the plurality of conversational responses received from the patient user according to an NLP model (Step 606). In accordance with certain aspects of the present disclosure, the NLP model is configured to extract one or more features from the plurality of conversational responses and cluster one or more text segments from the plurality of conversational responses according to the one or more features. Routine 600 may proceed by executing one or more steps or operations for assessing the patient user's risk of having an undiagnosed autoimmune condition according to a machine learning model (Step 608). In certain embodiments, routine 600 may assess the patient user's risk of having an undiagnosed autoimmune condition according to an output of the NLP model (i.e., without a separate analysis by the machine learning model). In accordance with certain aspects of the present disclosure, routine 600 may comprise a decision step 610 to determine whether the patient user is at risk (i.e., meets a specified risk threshold) based on an output of step 608. If NO (i.e., the patient user does not meet a specified risk threshold based on the plurality of conversational responses), then routine 600 proceeds by executing one or more steps or operations for providing a generative response to the patient user (e.g., via the conversational AI agent) stating that the patient user does not exhibit risk for the autoimmune condition (Step 612). If YES (i.e., the patient user satisfies a specified risk threshold based on the plurality of conversational responses), then routine 600 proceeds by executing one or more steps or operations for providing a generative response to the patient user (e.g., via the conversational AI agent) providing a risk profile or diagnostic analysis to the patient user (Step 614). In accordance with certain aspects of the present disclosure, routine 600 may proceed by executing one or more steps or operations for configuring one or more account parameters for the patient user within a care management application such that the patient user may proceed with one or more subsequent computerized interactions per an artificially intelligent diagnostic framework (Step 616).

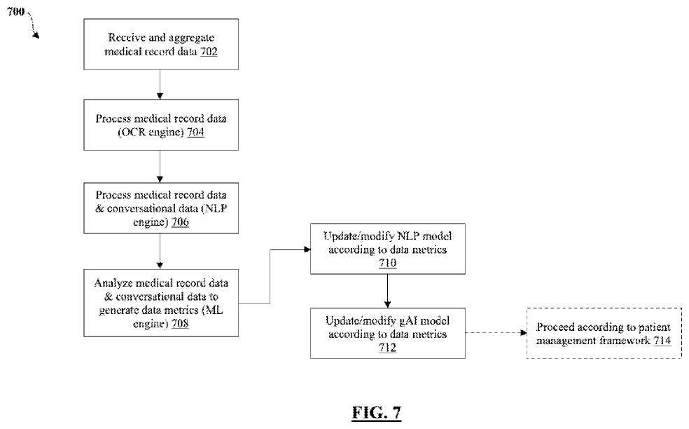

Referring now to FIG. 7, a process flow diagram of a routine 700 for an artificially intelligent system and method for prognostic evaluation of autoimmune disorders is shown. The operations of routine 700 may be performed in the order presented, in a different order, or simultaneously. Further, in some exemplary embodiments, some of the operations may be omitted, added, modified, skipped, or the like without departing from the scope of the invention. Routine 700 may be successive or sequential to one or more steps or operations of routine 600 (as shown in FIG. 6) and/or may comprise one or more sub-steps or sub-operations of routine 600. In accordance with certain aspects of the present disclosure, routine 700 may be embodied within one or more routines or operations of system 200, as shown and described in FIG. 2. Routine 700 may facilitate one or more steps of process flow 500, as shown in FIG. 5. In accordance with certain aspects of the present disclosure, routine 700 may comprise one or more steps or operations 702-714 for analyzing a plurality of medical record data and clinical conversation data to derive a patient phenotype or other patient-specific insights for use in management of an autoimmune condition in the patient user. In accordance with certain aspects of the present disclosure, the one or more steps or operations 702-714 are subsequent to step 616 in FIG. 6.

In accordance with certain aspects of the present disclosure, routine 700 may comprise one or more steps or operations for receiving and aggregating a plurality of medical record data (e.g., comprising a plurality of medical records for the patient user) at an application server or distributed computing environment (Step 702). Routine 700 may proceed by executing one or more steps or operations for processing the medical records (e.g., one or more scanned PDF or image files) with an OCR engine to extract (i.e., convert) a plurality of text data from the medical records (Step 704). Routine 700 may proceed by executing one or more steps or operations for processing the medical records data (i.e., converted text data) and clinical conversational data (e.g., the plurality of conversational responses received by the patient in FIG. 6 and/or one or more additional conversational responses) according to the natural language processing model (Step 706). In certain embodiments, the natural language processing model is configured to extract one or more features from the clinical conversational data and the medical record data and cluster one or more text segments from the clinical conversational data and the medical record data according to the one or more features. In accordance with certain aspects of the present disclosure, routine 700 may proceed by executing one or more steps or operations for analyzing the one or more text segments to determine one or more temporal or circumstantial association between the one or more text segments to generate one or more quantitative data metrics (Step 708). In accordance with certain aspects of the present disclosure, routine 700 may generate the one or more quantitative data metrics according to at least one machine learning engine. In certain embodiments, the quantitative data metrics comprise one or more prognostic values associated with the one or more text segments. In accordance with certain embodiments, step 708 may comprise one or more steps or operations for analyzing the one or more prognostic values for the one or more text segments to generate a prognostic assessment for at least one autoimmune disorder for the patient user. In accordance with certain aspects of the present disclosure, routine 700 may proceed by executing one or more steps or operations for updating and/or modifying (i.e., refining) one or more aspects of the NLP model according to the data metrics (Step 710). Routine 700 may further comprise one or more steps or operations for updating and/or modifying (i.e., refining) one or more aspects of the gAI model according to the data metrics (Step 712). In accordance with certain aspects of the present disclosure, routine 700 may proceed according to one or more steps or operations of an artificially intelligent patient management framework (Step 714).

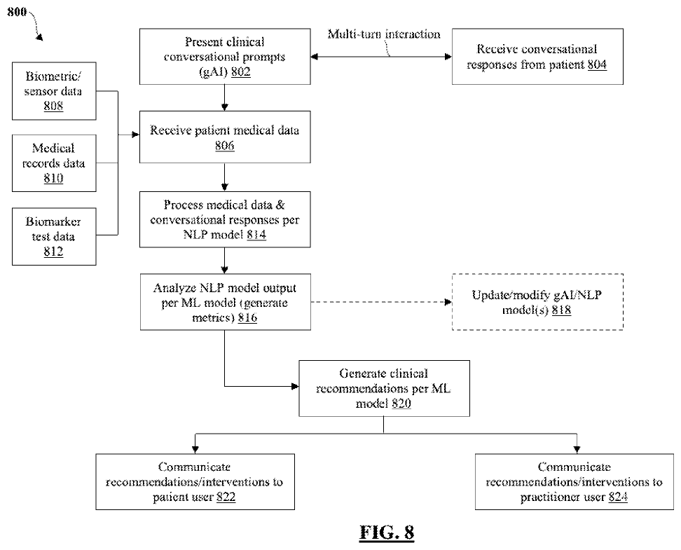

Referring now to FIG. 8, a process flow diagram of a routine 800 for an artificially intelligent system and method for prognostic evaluation of autoimmune disorders is shown. The operations of routine 800 may be performed in the order presented, in a different order, or simultaneously. Further, in some exemplary embodiments, some of the operations may be omitted, added, modified, skipped, or the like without departing from the scope of the invention. Routine 800 may be successive or sequential to one or more steps or operations of routine 600 and/or routine 700 (as shown, respectively, in FIGS. 6 and 7) and/or may comprise one or more sub-steps or sub-operations of routine 600 and/or routine 700. In accordance with certain aspects of the present disclosure, routine 800 may be embodied within one or more routines or operations of system 200, as shown and described in FIG. 2. Routine 800 may facilitate one or more steps of process flow 500, as shown in FIG. 5. In accordance with certain aspects of the present disclosure, routine 800 may comprise one or more steps or operations 802-824 for analyzing a plurality of medical record data and clinical conversation data to derive one or more interventions or clinical recommendations for treatment and/or management of the autoimmune condition in the patient user. In accordance with certain aspects of the present disclosure, the one or more steps or operations 802-824 are subsequent to step 714 in FIG. 7.

In accordance with certain aspects of the present disclosure, routine 800 may comprise one or more steps or operations for presenting a plurality of clinical conversational prompts to a patient user (e.g., according to the generative AI model) via the conversational AI agent (Step 802) and receiving a plurality of conversational responses from the patient user via the conversational AI agent (Step 804). The plurality of conversational responses comprises a plurality of clinical conversation data. In certain embodiments, steps 802-804 comprise a plurality of multi-turn (i.e., conversational) interactions between the patient user and the conversational AI agent. Routine 800 may proceed by executing one or more steps or operations for receiving a plurality of patient medical data via one or more data sources. In accordance with certain embodiments, the patient medical data may comprise a plurality of patient biometric data and/or physiological sensor data 808, medical record data 810, and/or blood biomarker testing data 812. Routine 800 may proceed by executing one or more steps or operations for processing the patient medical data and the clinical conversation data according to the NLP model to extract one or more features from the clinical conversational data and the medical record data and cluster one or more text segments from the clinical conversational data and the medical record data according to the one or more features (Step 814). In certain embodiments, step 814 may further comprise processing medical record data 810 and/or blood biomarker testing data 812 according to an OCR engine to convert or extract a plurality of text from the data for analysis by the NLP engine. Routine 800 may proceed by executing one or more steps or operations for analyzing one or more output of step 814 according to an ML engine to generate one or more quantitative metrics for the clinical conversation data based on one or more temporal or circumstantial associations between clinical conversation data and the patient medical data (Step 816). In accordance with certain embodiments, routine 800 may comprise one or more steps or operations for updating or modifying (i.e., refining) the gAI model(s) and/or the NLP model(s) based on an output of step 816 (Step 818). Routine 800 may proceed by executing one or more steps or operations for processing one or more outputs of step 816 to generate one or more clinical recommendations (Step 820). In accordance with certain embodiments, the one or more clinical recommendations may comprise one or more prognostic or diagnostic insights for the patient. In certain embodiments, the one or more clinical recommendations may comprise a patient phenotype comprising one or more personalized pathophysiological insights for the patent. In certain embodiments, the one or more clinical recommendations may comprise one or more recommended pharmaceutical intervention. In certain embodiments, the one or more clinical recommendations may comprise a predictive assessment of at least one pathophysiological event associated with the autoimmune disorder. In certain embodiments, the one or more clinical recommendations may comprise one or more behavioral or environmental recommendation for the patient user. In certain embodiments, the one or more clinical recommendations may comprise a clinical recommendation for at least one blood biomarker test for the patient. Routine 800 may proceed by executing one or more steps or operations for communicating the clinical recommendations and interventions to the patient user (Step 822) and the practitioner user (Step 824).

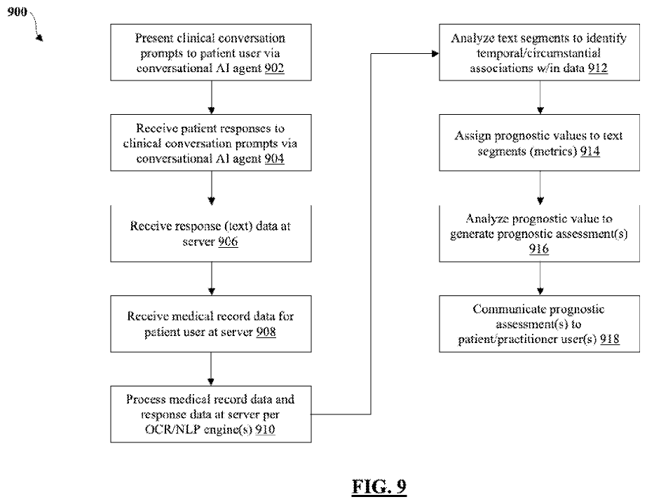

Referring now to FIG. 9, a process flow diagram of an artificially intelligent method 900 for prognostic evaluation of autoimmune disorders is shown. The steps or operations of method 900 may be performed in the order presented, in a different order, or simultaneously. Further, in some exemplary embodiments, some of the operations may be omitted, added, modified, skipped, or the like without departing from the scope of the invention. In accordance with certain aspects of the present disclosure, method 900 may be embodied within one or more routines or operations of system 200, as shown and described in FIG. 2. Method 900 may facilitate one or more steps of process flow 500, as shown in FIG. 5. In accordance with certain aspects of the present disclosure, method 900 may comprise one or more steps or operations 902-918 for analyzing a plurality of clinical conversation data and medical record data for a patient (e.g., according to one or more gAI, NLP and/or ML model) to identify one or more temporal or circumstantial associations within the data in order to generate a personalized prognostic evaluation of an autoimmune disorder for a patient user.

In accordance with certain aspects of the present disclosure, method 900 may comprise one or more steps or operations for presenting (e.g., with a conversational AI agent at one or more timepoints) a plurality of clinical conversational prompts to a patient user according to a gAI model (e.g., via a gAI engine executing on at least one server) (Step 902). Method 900 may further comprise one or more steps or operations for receiving (e.g., with the conversational AI agent at the one or more timepoints) a plurality of conversational responses from the patient user in response to the plurality of clinical conversational prompts (Step 904). Method 900 may further comprise one or more steps or operations for receiving (e.g., with the at least one server) a first set of text data comprising the plurality of conversational responses from the patient user (Step 906). Method 900 may further comprise one or more steps or operations for receiving (e.g., with the at least one server via at least one application programming interface) a first set of medical record data for the patient user (Step 908). In certain embodiments, the first set of medical record data for the patient user comprises at least one of blood biomarker test data and biometric measurement data. Method 900 may further comprise one or more steps or operations for processing (e.g., with the at least one server) the first set of text data and the first set of medical record data for the patient user according to a natural language processing model (Step 910). In certain embodiments, method 900 may comprise one or more steps or operations for processing the first set of medical record data via an OCR engine to convert one or more scanned or image files into a text searchable format. In certain embodiments, the natural language processing model is configured to extract one or more features from the first set of text data and the first set of medical record data. In certain embodiments, the natural language processing model is configured to cluster one or more text segments from the first set of text data and the first set of medical record data according to the one or more features. Method 900 may further comprise one or more steps or operations for analyzing, according to the natural language processing model, the one or more text segments to determine one or more temporal or circumstantial association between the one or more text segments and the blood biomarker test data and/or the biometric measurement data (Step 912). Method 900 may further comprise one or more steps or operations for assigning, according to at least one output of the natural language processing model, one or more prognostic values to the one or more text segments (Step 914). Method 900 may further comprise one or more steps or operations for analyzing (e.g., with the at least one server) the one or more prognostic values for the one or more text segments to generate a prognostic assessment for at least one autoimmune disorder for the patient user (Step 916). Method 900 may further comprise one or more steps or operations for providing (e.g., with the at least one server) the prognostic assessment to a practitioner user and/or a patient user via a graphical user interface of at least one client device (Step 918).

In accordance with certain aspects of the present disclosure, method 900 may further comprise one or more steps or operations for configuring (e.g., with the at least one server) a conversational AI model according to the one or more prognostic values for the one or more text segments. In certain embodiments, the conversational AI model may comprise a large language model. In certain embodiments, the plurality of clinical conversational prompts may comprise a plurality of generative prompts according to the conversational AI model. In certain embodiments, the prognostic assessment comprises a diagnostic assessment for the at least one autoimmune disorder. In certain embodiments, the prognostic assessment comprises a predictive assessment of at least one pathophysiological event associated with the at least one autoimmune disorder. In certain embodiments, the prognostic assessment comprises a clinical recommendation for at least one pharmaceutical intervention for the patient user. In certain embodiments, the prognostic assessment comprises a clinical recommendation for at least one blood biomarker test for the patient user. In certain embodiments, the biometric measurement data comprises data from at least one body-worn sensor of the patient user. In accordance with certain aspects of the present disclosure, method 900 may further comprise one or more steps or operations for modifying the conversational AI model to refine one or more clinical conversational prompts according to the one or more prognostic values for the one or more text segments in order to improve the relevance or specificity of one or more future conversational interactions between the patient user and the conversational AI agent.

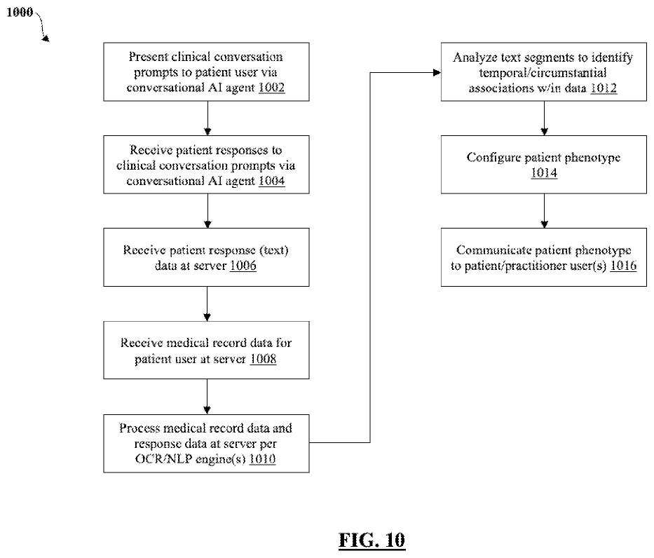

Referring now to FIG. 10, a process flow diagram of an artificially intelligent method 1000 for prognostic evaluation of autoimmune disorders is shown. The steps or operations of method 1000 may be performed in the order presented, in a different order, or simultaneously. Further, in some exemplary embodiments, some of the operations may be omitted, added, modified, skipped, or the like without departing from the scope of the invention. In accordance with certain aspects of the present disclosure, method 1000 may be embodied within one or more routines or operations of system 200, as shown and described in FIG. 2. Method 1000 may facilitate one or more steps of process flow 500, as shown in FIG. 5. In accordance with certain aspects of the present disclosure, method 1000 may comprise one or more steps or operations 1002-1016 for analyzing a plurality of clinical conversation data and medical record data for a patient (e.g., according to one or more gAI, NLP and/or ML model) to identify one or more temporal or circumstantial associations within the data in order to generate a personalized patent phenotype for a patient user to drive one or more pathophysiological insights for the management of an autoimmune disorder for the patient user.

In accordance with certain aspects of the present disclosure, method 1000 may comprise one or more steps or operations for presenting (e.g., with a conversational AI agent communicably engaged with a first server) a first set of clinical conversational prompts to a patient user according to a generative AI model (e.g., via a gAI engine executing on the first server) (Step 1002). Method 1000 may comprise one or more steps or operations for receiving (e.g., with the conversational AI agent) a first set of responses to the first set of clinical conversational prompts from the patient user (Step 1004). Method 1000 may comprise one or more steps or operations for receiving (e.g., with the first server) a first set of medical record data for the patient, wherein the first set of medical record data comprises at least one of blood biomarker test data and biometric measurement data for the patient user (Step 1006). Method 1000 may comprise one or more steps or operations for receiving (e.g., with at least one server) a first set of text data comprising the first set of responses to the first set of clinical conversational prompts from the patient user (Step 1008). Method 1000 may comprise one or more steps or operations for processing (e.g., with the at least one server) the first set of text data and the first set of medical record data according to a natural language processing model (e.g., via an NLP engine executing on the first server) (Step 1010). In accordance with certain aspects of the present disclosure, the natural language processing model is configured to extract one or more features from the first set of text data and the first set of medical record data. In accordance with said aspects, the natural language processing model is configured to cluster one or more text segments from the first set of text data and the first set of medical record data according to the one or more features. Method 1000 may comprise one or more steps or operations for analyzing, according to the natural language processing model, the one or more text segments to determine one or more temporal or circumstantial association between the one or more text segments and the blood biomarker test data and/or the biometric measurement data (Step 1012). Method 1000 may comprise one or more steps or operations for configuring, according to at least one output of the natural language processing model, a patient phenotype for the patient user according to the one or more temporal or circumstantial association between the one or more text segments and the blood biomarker test data and/or the biometric measurement data (Step 1014). In accordance with certain aspects of the present disclosure, the patient phenotype comprises one or more personalized insights for the patient user comprising, for example, one or more symptom, marker, and pathology trigger for an autoimmune disorder for the patient user. Method 1000 may comprise one or more steps or operations for providing, with the at least one server, the patient phenotype for the patient user to a practitioner user and/or the patient user via a graphical user interface of one or more client device (Step 1016).

In accordance with certain aspects of the present disclosure, method 1000 may further comprise one or more steps or operations for configuring (e.g., with the at least one server) the generative AI model according to the patient phenotype. Method 1000 may further comprise one or more steps or operations for presenting (e.g., with the conversational AI agent) a second set of clinical conversational prompts to a patient user according to the generative AI model. In certain embodiments, the second set of clinical conversational prompts are configured according to the patient phenotype (e.g., per the generative AI model). In accordance with certain aspects of the present disclosure, at least one clinical prompt in the second set of clinical conversational prompts is different than the first set of clinical conversational prompts. Method 1000 may further comprise one or more steps or operations for receiving (e.g., with the conversational AI agent) a second set of responses to the second set of clinical conversational prompts from the patient user. Method 1000 may further comprise one or more steps or operations for receiving (e.g., with the first server) a second set of medical record data for the patient, wherein the second set of medical record data comprises a second set of blood biomarker test data and/or a second set of biometric measurement data for the patient user. Method 1000 may further comprise one or more steps or operations for receiving (e.g., with the at least one server) a second set of text data comprising the second set of responses to the second set of clinical conversational prompts from the patient user. Method 1000 may further comprise one or more steps or operations for processing (e.g., with the at least one server) the second set of text data according to the natural language processing model. In certain embodiments, the natural language processing model is configured to extract one or more features from the second set of text data according to the patient phenotype. In certain embodiments, the natural language processing model is configured to cluster one or more text segments from the second set of text data according to the one or more features. Method 1000 may further comprise one or more steps or operations for processing (e.g., with the at least one server) the second set of text data and the second set of medical record data according to the natural language processing model. In accordance with certain aspects of the present disclosure, the natural language processing model is configured to extract one or more features from the second set of text data and the second set of medical record data according to the patient phenotype. In certain embodiments, the natural language processing model is configured to cluster one or more text segments from the second set of text data and the second set of medical record data according to the one or more features. Method 1000 may further comprise one or more steps or operations for analyzing (e.g., according to a machine learning model) the one or more text segments to generate a prognostic assessment for the autoimmune disorder for the patient user. Method 1000 may further comprise one or more steps or operations for providing (e.g., with the at least one server) the prognostic assessment to the patient user via a graphical user interface of an end user device associated with the patient user and/or the practitioner user via the graphical user interface of the client device. Method 1000 may further comprise one or more steps or operations for updating or modifying the patient phenotype according to at least one output of the natural language processing model. In certain embodiments, the prognostic assessment comprises one or more recommended actions for management of the autoimmune disorder for the patient user.

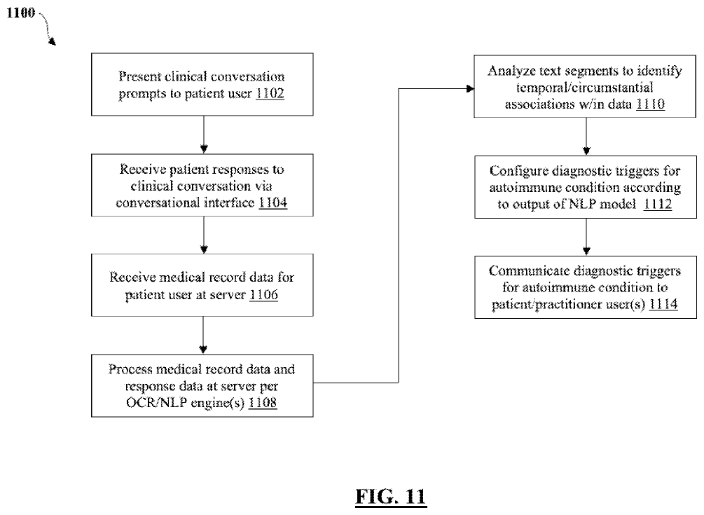

Referring now to FIG. 11, a process flow diagram of an artificially intelligent method for prognostic evaluation of autoimmune disorders is shown. The steps or operations of method 1100 may be performed in the order presented, in a different order, or simultaneously. Further, in some exemplary embodiments, some of the operations may be omitted, added, modified, skipped, or the like without departing from the scope of the invention. In accordance with certain aspects of the present disclosure, method 1100 may be embodied within one or more routines or operations of system 200, as shown and described in FIG. 2. Method 1100 may facilitate one or more steps of process flow 500, as shown in FIG. 5. In accordance with certain aspects of the present disclosure, method 1100 may comprise one or more steps or operations 1102-1114 for analyzing a plurality of clinical conversation data and medical record data for a patient (e.g., according to one or more gAI, NLP and/or ML models) to identify one or more diagnostic triggers for an autoimmune disease from the clinical conversational data in order to improve diagnostic accuracy and reduce time to an autoimmune disease diagnosis.

In accordance with certain aspects of the present disclosure, method 1100 may comprise one or more steps or operations for presenting (e.g., with at least one server communicably engaged with a first client device) a plurality of clinical conversation prompts to a patient user at a user interface of the first client device according to a generative AI model (e.g., via a gAI engine executing on the at least one server) (Step 1102). Method 1100 may further comprise one or more steps or operations for receiving (e.g., with the at least one server via the first client device) a plurality of user-generated responses to the plurality of clinical conversation prompts from the patient user (e.g., via the conversational AI agent), the plurality of user-generated responses to the plurality of clinical conversation prompts comprising a first set of clinical conversation data (Step 1104). Method 1100 may further comprise one or more steps or operations for receiving (e.g., with the at least one server) a first set of medical record data for the patient user, wherein the first set of medical record data comprises at least one of blood biomarker test data and biometric measurement data for the patient user (Step 1106). Method 1100 may further comprise one or more steps or operations for processing (e.g., with the at least one server) the first set of clinical conversation data and the first set of medical record data according to a natural language processing model (e.g., via an NLP engine executing on the at least one server) (Step 1108). In certain embodiments, the natural language processing model is configured to extract one or more features from the first set of clinical conversation data and the first set of medical record data. The natural language processing model may be configured to cluster one or more text segments from the first set of clinical conversation data and the first set of medical record data according to the one or more features. Method 1100 may further comprise one or more steps or operations for analyzing, according to at least one output of the natural language processing model, the one or more text segments to determine one or more temporal or circumstantial association between the one or more text segments and the blood biomarker test data and/or the biometric measurement data (Step 1110). Method 1100 may further comprise one or more steps or operations for configuring, according to the at least one output of the natural language processing model, one or more diagnostic triggers for the patient user according to the one or more temporal or circumstantial association between the one or more text segments and the blood biomarker test data and/or the biometric measurement data (Step 1112). In certain embodiments, step 1112 may comprise one or more steps or operations for analyzing the at least one output of the natural language processing model according to at least one machine learning model (e.g., via at least one ML engine executing on the at least one server). Method 1100 may further comprise one or more steps or operations for communicating (e.g., with the at least one server via a network interface) the one or more diagnostic triggers to the patient user via the first client device and/or a practitioner user via a second client device (Step 1114).

In accordance with certain aspects of the present disclosure, method 1100 may further comprise one or more steps or operations for configuring (e.g., with the at least one server) the natural language processing model according to the one or more diagnostic triggers. In certain embodiments, the plurality of clinical conversation prompts are configured according to a generative AI model. In said embodiments, the generative AI model may comprise a large language model. Method 1100 may further comprise one or more steps or operations for configuring (e.g., with the at least one server) the generative AI model according to the one or more diagnostic triggers. Method 1100 may further comprise one or more steps or operations for presenting (e.g., with the at least one server communicably engaged with the first client device) a second or subsequent plurality of clinical conversation prompts to the patient user at the user interface of the first client device. Method 1100 may further comprise one or more steps or operations for receiving (e.g., with the at least one server via the first client device) a second or subsequent plurality of user-generated responses to the second or subsequent plurality of clinical conversation prompts from the patient user, the second or subsequent plurality of user-generated responses to the second or subsequent plurality of clinical conversation prompts comprising a second or subsequent set of clinical conversation data. Method 1100 may further comprise one or more steps or operations for processing (e.g., with the at least one server) the second or subsequent set of clinical conversation data according to the natural language processing model to extract one or more features from the second or subsequent set of clinical conversation data according to the one or more diagnostic triggers. Method 1100 may further comprise one or more steps or operations for analyzing, according to a machine learning model, at least one output of the natural language processing model to identify at least one diagnostic trigger from the second or subsequent set of clinical conversation data. Method 1100 may further comprise one or more steps or operations for communicating (e.g., with the at least one server) the at least one diagnostic trigger from the second or subsequent set of clinical conversation data to the practitioner user via the graphical user interface of the second client device. Method 1100 may further comprise one or more steps or operations for communicating (e.g., with the at least one server) the at least one diagnostic trigger from the second or subsequent set of clinical conversation data to the patient user at the user interface of the first client device. Method 1100 may further comprise one or more steps or operations for generating, according to the machine learning model, at least one clinical recommendation for management of the autoimmune disorder according to the at least one diagnostic trigger from the second or subsequent plurality of user-generated responses. Method 1100 may further comprise one or more steps or operations for communicating (e.g., with the at least one server) the at least one clinical recommendation for management of the autoimmune disorder to the practitioner user via the graphical user interface of the second client device. Method 1100 may further comprise one or more steps or operations for communicating (e.g., with the at least one server) the at least one clinical recommendation for management of the autoimmune disorder to the patient user at the user interface of the first client device.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random-access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general-purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational phases to be performed on the computer or other programmable apparatus to produce a computer-implemented process such that the code portions which execute on the computer or other programmable apparatus provide phases for implementing the functions/acts specified in the flowchart and/or block diagram block(s). Alternatively, computer program implemented phases or acts may be combined with operator or human implemented phases or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be performed in an order other than the order illustrated, may be combined or divided, or may be performed simultaneously. It will also be understood that the blocks of the block diagrams illustrate, in some embodiments, merely conceptual delineations between systems, and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1-98. (canceled)

99. A computer-implemented method for prognostic evaluation of autoimmune disorders comprising:
presenting, with a conversational AI agent at one or more timepoints, a plurality of clinical conversational prompts to a patient user;
receiving, with the conversational AI agent at the one or more timepoints, a plurality of conversational responses from the patient user in response to the plurality of clinical conversational prompts;
receiving, with at least one server, a first set of text data comprising the plurality of conversational responses from the patient user;
receiving, with the at least one server via at least one application programming interface, a first set of medical record data for the patient user,
wherein the first set of medical record data for the patient user comprises at least one of blood biomarker test data and biometric measurement data;
processing, with the at least one server, the first set of text data and the first set of medical record data for the patient user according to a natural language processing model,
wherein the natural language processing model is configured to extract one or more features from the first set of text data and the first set of medical record data,
wherein the natural language processing model is configured to cluster one or more text segments from the first set of text data and the first set of medical record data according to the one or more features;
analyzing, according to the natural language processing model, the one or more text segments to determine one or more temporal or circumstantial association between the one or more text segments and the blood biomarker test data and/or the biometric measurement data;
assigning, according to at least one output of the natural language processing model, one or more prognostic values to the one or more text segments;
analyzing, with the at least one server, the one or more prognostic values for the one or more text segments to generate a prognostic assessment for at least one autoimmune disorder for the patient user; and
providing, with the at least one server, the prognostic assessment to a practitioner user via a graphical user interface of a client device.

100. The computer-implemented method of claim 99 further comprising configuring, with the at least one server, a conversational AI model according to the one or more prognostic values for the one or more text segments.

101. The computer-implemented method of claim 100 wherein the conversational AI model comprises a large language model.

102. The computer-implemented method of claim 100 wherein the plurality of clinical conversational prompts comprise a plurality of generative prompts according to the conversational AI model.

103. The computer-implemented method of claim 99 wherein the prognostic assessment comprises a diagnostic assessment for the at least one autoimmune disorder.

104. The computer-implemented method of claim 99 wherein the prognostic assessment comprises a predictive assessment of at least one pathophysiological event associated with the at least one autoimmune disorder.

105. The computer-implemented method of claim 99 wherein the prognostic assessment comprises a clinical recommendation for at least one pharmaceutical intervention for the patient user.

106. The computer-implemented method of claim 99 wherein the prognostic assessment comprises a clinical recommendation for at least one blood biomarker test for the patient user.

107. The computer-implemented method of claim 99 wherein the biometric measurement data comprises data from at least one body-worn sensor of the patient user.

108. The computer-implemented method of claim 100 further comprising modifying, via the conversational AI model, the plurality of clinical conversational prompts according to the one or more prognostic values for the one or more text segments.

109. A system for prognostic evaluation of autoimmune disorders comprising:
a first client device associated with a patient user, wherein the first client device comprises a first graphical display and a first input/output device;
a second client device associated with a practitioner user, wherein the second client device comprises a second graphical display and a second input/output device;
an application server communicably engaged with the first client device and the second client device via a network communications interface, wherein the application server comprises a generative AI engine and a natural language processing engine,
wherein the application server comprises at least one processor and a non-transitory computer readable medium having instructions stored thereon that, when executed by the at least one processor, cause the processor to perform one or more operations, the one or more operations comprising:
presenting, according to a generative AI model executing on the generative AI engine, a plurality of clinical conversational prompts at the first client device;
receiving a plurality of conversational responses from the patient user in response to the plurality of clinical conversational prompts, the plurality of conversational responses comprising a first set of text data;
receiving, via at least one application programming interface, a first set of medical record data for the patient user,
wherein the first set of medical record data for the patient user comprises at least one of blood biomarker test data and biometric measurement data;
processing the first set of text data and the first set of medical record data for the patient user according to a natural language processing model executing on a natural language processing engine,
wherein the natural language processing model is configured to extract one or more features from the first set of text data and the first set of medical record data,
wherein the natural language processing model is configured to cluster one or more text segments from the first set of text data and the first set of medical record data according to the one or more features;
analyzing, according to the natural language processing model, the one or more text segments to determine one or more temporal or circumstantial association between the one or more text segments and the blood biomarker test data and/or the biometric measurement data;
assigning, according to the natural language processing model, one or more prognostic values to the one or more text segments;
analyzing the one or more prognostic values for the one or more text segments to generate a prognostic assessment for at least one autoimmune disorder for the patient user; and
providing the prognostic assessment to the first client device and/or the second client device via the network communications interface.

110. The system of claim 109 further comprising at least one biometric sensor configured to collect the biometric measurement data for the patient user.

111. The system of claim 110 wherein the at least one biometric sensor comprises a body-worn sensor configured to continuously collect the biometric measurement data when worn by the patient user.

112. The system of claim 111 wherein the at least one biometric sensor is communicably engaged with the first client device to communicate a plurality of sensor inputs comprising the biometric measurement data to the first client device in real-time.

113. The system of claim 109 wherein the generative AI model comprises a large language model.

114. The system of claim 109 wherein the biometric measurement data comprises at least one data type selected from the group consisting of electrocardiogram data, heartrate data, heartrate variability data, sleep data, and activity data.

115. The system of claim 109 wherein the prognostic assessment comprises a diagnostic assessment for the at least one autoimmune disorder.

116. The system of claim 109 wherein the prognostic assessment comprises a predictive assessment of at least one pathophysiological event associated with the at least one autoimmune disorder.

117. The system of claim 109 wherein the prognostic assessment comprises a clinical recommendation for at least one pharmaceutical intervention for the patient user.

118. The system of claim 109 wherein the prognostic assessment comprises a clinical recommendation for at least one blood biomarker test for the patient user.
